SEQUENCE LISTING (9 sequences)

SEQ ID NO: 1 - RNA, 21 nt, Artificial Sequence
  Synthetic RNA
gugccgggauguugcuauaga

SEQ ID NO: 2 - RNA, 21 nt, Artificial Sequence
  Synthetic RNA
aacacggcccuacaacgauau

SEQ ID NO: 3 - RNA, 21 nt, Artificial Sequence
  Synthetic RNA
caaggagcagggacaaguuac

SEQ ID NO: 4 - RNA, 21 nt, Artificial Sequence
  Synthetic RNA
aaguuccucgucccuguucaa

SEQ ID NO: 5 - RNA, 21 nt, Artificial Sequence
  Synthetic RNA
gaugaggaucguuucgcauga

SEQ ID NO: 6 - RNA, 21 nt, Artificial Sequence
  Synthetic RNA
uccuacuccuagcaaagcgua

SEQ ID NO: 7 - RNA, 23 nt, Artificial Sequence
  Synthetic RNA
aacacggcccuacaacgauaucu

SEQ ID NO: 8 - RNA, 23 nt, Artificial Sequence
  Synthetic RNA
aaguuccucgucccuguucaaug

SEQ ID NO: 9 - RNA, 23 nt, Artificial Sequence
  Synthetic RNA
uccuacuccuagcaaagcguacu

(19) United States
(12) Patent Application Publication
(10) Pub. No.: US 2004/0126791 A1
(43) Pub. Date: Jul. 1, 2004

(54) Compositions and methods for treating trail-resistant cancer cells

(75) Inventors: Harald Wajant, Kist (DE); Klaus Pfizenmaier, Tiefenbronn (DE); Stefan Limmer, Kulmbach (DE); Roland Kreutzer, Weidenberg (DE); Hans-Peter Vornlocher, Bayreuth (DE)

Correspondence Address: PALMER & DODGE, LLP, KATHLEEN M. WILLIAMS, 111 HUNTINGTON AVENUE, BOSTON, MA, 02199, US

(73) Assignee: Ribopharma AG

(21) Appl. No.: 10/666,458

(22) Filed: Sep. 19, 2003

Related U.S. Application Data
(63) Continuation-in-part of application No. PCT/EP02/11968, filed on Oct. 25, 2002.

(30) Foreign Application Priority Data
Jan. 9, 2002 (WO) PCT/EP02/00152
Jan. 9, 2002 (WO) PCT/EP02/00151
Oct. 26, 2001 (DE) 101 55 280.7
Nov. 29, 2001 (DE) 101 58 411.3
Jul. 9, 2002 (DE) 102 30 996.5

(51) Int. Cl. C12Q 1/68; C07H 21/02; A61K 48/00

(52) U.S. Cl. 435/6; 435/69.1; 435/320.1; 435/325; 530/350; 536/23.1; 514/44

(57) ABSTRACT

The present invention relates to a double-stranded ribonucleic acid (dsRNA) for inhibiting the expression of a cellular FLICE-like inhibitory protein (cFLIP) gene, comprising a complementary RNA strand which is substantially identical to at least a part of a cFLIP gene. The invention also relates to a pharmaceutical composition comprising the dsRNA together with a pharmaceutically acceptable carrier; methods for inhibiting the expression of a cFLIP gene in a cell, methods for improving the effectiveness of an apoptosis-inducing drug, and methods for treating cancer using the pharmaceutical composition.

### RELATED APPLICATIONS

This application is a continuation-in-part of International Application No. PCT/EP02/11968, which designated the United States and was filed on Oct. 25, 2002, which claims the benefit of German Patent No. 101 55 280.7, filed on Oct. 26, 2001, German Patent No. 101 58 411.3, filed on Nov. 29, 2001, EP Patent No. PCT/EP02/00152, filed on Jan. 9, 2002, EP Patent No. PCT/EP02/00151, filed Jan. 9, 2002, and German Patent No. 102 30 996.5, filed on Jul. 9, 2002. The entire teachings of the above applications are incorporated herein by reference.

### FIELD OF THE INVENTION

This invention relates to compositions and methods for inhibiting the expression of cellular FLICE-like inhibitory protein (cFLIP), improving the effectiveness of apoptosis-inducing drugs, and treating cancer using double-stranded ribonucleic acid (dsRNA).

### BACKGROUND OF THE INVENTION

Programmed cell death (PCD), or apoptosis, has become the subject of intense study in recent years because of its recognized association with various physiological processes, including embryogenesis, tissue regeneration, differentiation, development of the immune system, autoimmunity, elimination of diseased cells, and the maintenance of tissue homeostasis (Thompson, Science (1995), 267:1456-1462). For a general review of apoptosis, see Tomei et al., Apoptosis: The Molecular Basis of Cell Death, Cold Spring Harbor Press, N.Y. (1991); Tomei et al., Apoptosis II: The Molecular Basis of Apoptosis in Disease, Cold Spring Harbor Press, N.Y. (1994); and Duvall et al., Immun. Today (1986) 4:115-119.

In addition to its role in normal physiological processes, apoptosis also occurs in response to a variety of external stimuli, including growth factor deprivation, exposure to free-radicals and cytotoxic lymphokines, infection by some viruses, radiation and most chemotherapeutic agents. Although normally subject to cellular regulatory mechanisms, dysregulation of apoptosis also can occur and is observed, for example, in some types of cancer cells and in neurodegenerative diseases involving premature death of neurons. Induction of apoptosis also occurs in the pathophysiology of the disease process, for example, in immune-based eradication of viral infections, wherein host cells undergo immune cell attack resulting in apoptosis.

Many of the proteins involved in programmed cell death have been identified and characterized. For example, an entire family of mammalian proteases have been identified, including at least 14 different members of the caspase family (cystein-containing aspartate-specific protease) (Wyllie, J. Pathol. (1987) 153:313-316; Thornberry, et al., Science (1998) 281:1312-1316; and Cohen, Biochem. J. (1997) 326:1-16). The caspases involved in apoptosis have been divided into two groups based on their structure and function. The long prodomain “initiator” caspases include caspases 2, 8, 9, and 10. These initiator caspases contain an amino-terminal domain that is cleaved during activation. The short prodomain “executioner” caspases include caspases 3, 6, and 7. These executioner caspases are activated by the initiator caspases. Once activated, the executioner caspases cleave numerous cellular proteins, which ultimately results in cell death.

The tumor necrosis factor-related apoptosis-inducing ligand (TRAIL or Apo2L) has been shown to be a selective and potent inducer of apoptosis in cancer cells (but not normal cells) upon binding to either of two pro-apoptotic TRAIL receptors, TRAIL R1 (DR4) (Pan, et al., Science (1997) 276:111-113) or TRAIL R2 (KILLER/DR5) (Wu, et al., Nat. Genet. (1997) 17:141-143; Pan, et al., Science (1997) 277:815-818; and Walczak, et al., EMBO J. (1997) 16:5386-5397). Activation of the pro-apoptotic death receptors by TRAIL induces the formation of death inducing signaling complex (DISC), which consists of receptor FADD as an adaptor (Kischkel, et al., Immunity (2000) 12:611-620; and Kuang, et al., J. Biol. Chem. (2000) 275:25065-25068), and caspase 8 as an initiator caspase. Once DISC is formed, caspase 8 is auto-processed and activated by induced proximity (Medema, et al., EMBO J. (1997) 16:2794-2804; and Muzio, et al., J. Biol. Chem. (1998) 273:2926-2930).

TRAIL has generated significant interest as a potential cancer therapeutic (French, et al., Nat. Med. (1999) 5:146-147) because of its selective targeting of cancer cells; most normal cells appear to be resistant to TRAIL (Ashkenazi, et al., Science (1998) 281:1305-1308; and Walczak, et al., Nat. Med. (1999) 5:157-163). Systemic administration of TRAIL has proven to be safe and effective at killing breast or colon xenografted tumors and prolonging survival in mice (Walczak, et al., 1999). Although TRAIL can specifically kill many types of cancer cells, many others display TRAIL-resistance (Kim, et al., Clin. Cancer Res. (2000) 6:335-346; and Zhang, et al., Cancer Res. (1999) 59:2747-2753).

Numerous mechanisms have been identified as responsible for TRAIL-resistance. Such mechanisms exist at a number of levels, including at the receptor level, mitochondria level, post-mitochondria level, and at the DISC level. For example, loss of caspase 8 expression (Teitz, et al., Nat. Med. (2000) 6:529-535; and Griffith, et al., J. Immunol. (1998) 161:2833-2840) or high expression of the cellular FLICE inhibitor protein (cFLIP) (Kim, et al., 2000; Zhang, et al., 1999; and Kataoka, et al., J. Immunol. (1998) 161:3936-3942) make cancer cells resistant to TRAIL. W. C. Yeh, et al. have shown that cFLIP-deficient embryonic mouse fibroblasts are particularly sensitive to receptor-mediated apoptosis (Yeh, et al., Immunity (2000) 12:533-642). Several splice variants of cFLIP are known, including a short splice variant, cFLIP-S, and a longer splice variant, cFLIP-L. Bin, L., et al., FEBS Lett. (2002) 510(1-2):37-40 show that cFLIP-deficient embryonic mouse fibroblasts become resistant to TRAIL-induced apoptosis as a result of retroviral-mediated transduction of cFLIP-S.

Although TRAIL represents a particularly promising candidate for tumor-selective death receptor activation (i.e., induces apoptosis preferentially in tumor cells but not in normal tissues), many cancer cells are resistant to apoptosis-inducing drugs, as discussed above. As a result, treatment with such drugs often requires co-treatment with irradiation and/or cytotoxic chemicals to achieve a therapeutic effect. However, both radiation and chemotherapy have significant side effects, and are generally avoided if possible.

Double-stranded RNA molecules (dsRNA) have been shown to block gene expression in a highly conserved regulatory mechanism known as RNA interference (RNAi). Briefly, the RNAse III Dicer processes dsRNA into small interfering RNAs (siRNA) of approximately 22 nucleotides, which serve as guide sequences to induce target-specific mRNA cleavage by an RNA-induced silencing complex RISC (Hammond, S. M., et al., Nature (2000) 404:293-296). When administered to a cell or organism, exogenous dsRNA has been shown to direct the sequence-specific degradation of endogenous messenger RNA (mRNA) through RNAi. This phenomenon has been observed in a variety of organism, including mammals (see, e.g., WO 00/44895, Limmer; and DE 101 00 586 C1, Kruetzer, et al.). Although now recognized as a promising candidate for selectively inhibiting expression of disease-associated genes, such as oncogenes, dsRNA has never been suggeseted as a means for sensitizing tumor cells for drug-induced apoptosis.

Thus, a need exists for an agent that can selectively and efficiently sensitize tumor cells for apoptosis-inducing drugs such as TRAIL, without also sensitizing the surrounding normal cells. Such an agent would be useful for reducing or preventing the drug resistance commonly associated with the use of receptor-mediated apoptotic cancer drugs, thus improving their effectiveness and eliminating the need for secondary therapies.

### SUMMARY OF THE INVENTION

The present invention discloses double-stranded ribonucleic acid (dsRNA), as well as compositions and methods for inhibiting the expression of a cellular FLICE-like inhibitory protein (cFLIP) using the dsRNA. The dsRNA of the invention comprises an RNA strand (the complementary strand) having a region which is complementary to at least a portion of an RNA transcript of a cFLIP gene. The present invention also discloses compositions and methods for improving the effectiveness of a bioactive substance that induces receptor-mediated apoptosis in tumor cells, as well as methods for treating cancer using the inventive compositions.

In one aspect, the invention relates to double-stranded ribonucleic acid (dsRNA) for inhibiting the expression of a cFLIP gene in a cell. The dsRNA comprises a complementary RNA strand having a nucleotide sequence which is complementary to at least a part of the cFLIP gene and a second (sense) RNA strand. The dsRNA further comprises a nucleotide overhang of 1 to 4, preferably 2 or 3, nucleotides in length, and a blunt end, wherein the nucleotide overhang is at the 3′-terminus of the complementary RNA strand, and the dsRNA is blunt-ended at the 5′-end of the complementary RNA strand. The nucleotide sequence may be less than 25 nucleotides in length, 19 to 24 nucleotides in length, 20 to 24 nucleotides in length, 21 to 23 nucleotides in length, or 22 or 23 nucleotides in length. The complementary RNA strand may be less than 30 nucleotides in length, less than 25 nucleotides in length, 21 to 24 nucleotides in length, or 23 nucleotides in length. The dsRNA may further comprise a second (sense) RNA strand. The complementary RNA strand may be 23 nucleotides in length and the second RNA strand may be 21 nucleotides in length. The complementary RNA strand may have a 3′-end and a 5′-end, wherein the 3′-end has a nucleotide overhang of 2 nucleotides in length, and wherein the blunt end of the dsRNA is at the 5′-end of the complementary RNA strand. The nucleotide sequence of the complementary RNA strand may be complementary to a primary or processed RNA transcript of the cFLIP gene. The complementary RNA strand may comprise SEQ ID NO:2 and the second RNA strand may comprise SEQ ID NO:1; the complementary RNA strand may comprise SEQ ID NO:4 and the second RNA strand may comprise SEQ ID NO:3; the complementary RNA strand may comprise SEQ ID NO:7 and the second RNA strand may comprise SEQ ID NO:1; or the complementary RNA strand may be comprise SEQ ID NO:8 and the second RNA strand may comprise SEQ ID NO:3. The cell may be a tumor cell. The tumor cell may be resistant to treatment with an apoptosis-inducing drug, such as a TNF-related apoptosis-inducing ligand (TRAIL).

In another aspect, the invention relates to a method for inhibiting the expression of a cFLIP gene in a cell. The method comprises introducing a dsRNA, as described above, into the cell, and maintaining the cell for a time sufficient to obtain degradation of a mRNA transcript of the cFLIP gene. The cell may be a tumor cell. The tumor cell may be resistant to treatment with an apoptosis-inducing drug, such as TRAIL.

In yet another aspect, the invention relates to a pharmaceutical composition for improving the effectiveness of an apoptosis-inducing drug in a mammal. The composition comprises a dsRNA, as described above, and a pharmaceutically acceptable carrier. The apoptosis-inducing drug may be a tumor necrosis factor (TNF) or a TNF-related ligand. The TNF-related ligand may be a TRAMP ligand, a CD95 ligand, a TNFR-1 ligand, or TRAIL. The mammal may be a human. The dosage unit of dsRNA may be less than 5 milligram of dsRNA per kilogram body weight of the mammal; in a range of 0.01 to 2.5 milligrams, 0.1 to 200 micrograms, or 0.1 to 100 micrograms per kilogram body weight of the mammal; less than 50 micrograms per kilogram body weight of the mammal; or less than 25 micrograms per kilogram body weight of the mammal. The pharmaceutically acceptable carrier may be an aqueous solution, such as phosphate buffered saline, or it may comprise a micellar structure, such as a liposome, capsid, capsoid, polymeric nanocapsule, or polymeric microcapsule. The pharmaceutical composition may be formulated for inhalation or oral ingestion, or it may be formulated for infusion or injection, such as intravenous, intraparenteral, or intratumoral infusion or injection.

In yet another aspect, the invention relates to a pharmaceutical composition for inhibiting the expression of a cFLIP gene in a mammal. The composition comprises a dsRNA and a pharmaceutically acceptable carrier, as described above.

In still another aspect, the invention relates to a method for improving the effectiveness of a bioactive substance that induces receptor-mediated apoptosis in a tumor cell in a mammal. The method comprises administering a pharmaceutical composition comprising a dsRNA and a pharmaceutically acceptable carrier, as described above. The bioactive substance may be a tumor necrosis factor (TNF) or a TNF-related ligand. The TNF-related ligand may be a TRAMP ligand, a CD95 ligand, a TNFR-1 ligand, or TRAIL.

In still a further aspect, the invention relates to a method for treating cancer in a mammal. The method comprises administering a pharmaceutical composition comprising a dsRNA and a pharmaceutically acceptable carrier, as described above, and a pharmaceutical composition comprising a bioactive substance that induces receptor-mediated apoptosis in a tumor cell. The bioactive substance may be a tumor necrosis factor (TNF) or a TNF-related ligand. The TNF-related ligand may be a TRAMP ligand, a CD95 ligand, a TNFR-1 ligand, or TRAIL. The method may comprise administering the dsRNA and the bioactive substance together in one composition.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

FIG. 1 shows the sensitizing effect on TRAIL-R1- and TRAIL-R2-mediated apoptosis in SV80 cells after transfection with a dsRNA comprising a nucleotide sequence complementary to at least a portion of a cFLIP gene, as compared to control SV80 cells.

FIG. 2 shows the sensitizing effect on TRAIL-R1- and TRAIL-R2-mediated apoptosis in KB cells after transfection with a dsRNA comprising a nucleotide sequence complementary to at least a portion of a cFLIP gene, as compared to control KB cells.

### DETAILED DESCRIPTION OF THE INVENTION

The present invention discloses double-stranded ribonucleic acid (dsRNA), as well as compositions and methods for inhibiting the expression of a cellular FLICE-like inhibitory protein (cFLIP) using the dsRNA. The dsRNA of the invention comprises an RNA strand (the complementary strand) having a region which is complementary to at least a portion of an RNA transcript of a cFLIP gene. The present invention also discloses compositions and methods for improving the effectiveness of a bioactive substance that induces receptor-mediated apoptosis in tumor cells, as well as methods for treating cancer using the inventive compositions.

The dsRNA of the invention comprises an RNA strand (the complementary strand) which is complementary to at least a portion of an RNA transcript of a cFLIP gene. The use of these dsRNAs enables the targeted degradation of mRNAs of genes that are implicated in resistance to substances that induce receptor-mediated apoptosis in tumor cells. Using cell-based assays, the present inventors have demonstrated that very low dosages of these dsRNA can specifically and efficiently mediate RNAi, resulting in significant inhibition of expression of the cFLIP gene. The dsRNA affects receptor-mediated apoptosis to such an extent that there is a noticeable reduction in the number of living cells in dsRNA-transfected human tumor cell lines. Thus, the present invention encompasses these dsRNAs and compositions comprising dsRNA and their use for specifically silencing cFLIP genes whose protein products are implicated in the resistance of cancer cells to treatment with apoptosis-inducing drugs. Moreover, the dsRNAs of the invention have no apparent effect on neighboring normal cells. Thus, the methods and compositions of the present invention comprising these dsRNAs are useful for improving the efficiency of apoptosis-inducing drugs and for treating cellular proliferative and/or differentiation disorders, such as cancer.

The following detailed description discloses how to make and use the dsRNA and compositions containing dsRNA to inhibit the expression of cFLIP genes, as well as compositions and methods for improving the effectiveness of bioactive substances that induce receptor-mediated apoptosis in cancer cells, and for treating proliferative diseases and disorders such as cancer. The pharmaceutical compositions of the present invention comprise a dsRNA having an RNA strand comprising a complementary region which is complementary to at least a portion of an RNA transcript of a cFLIP gene, together with a pharmaceutically acceptable carrier. The cFLIP gene may be any mutant form or variation of the wild-type cFLIP gene, such as a short splice variant, cFLIP-S, or a longer splice variant, cFLIP-L.

Accordingly, certain aspects of the present invention relate to pharmaceutical compositions comprising the dsRNA of the present invention together with a pharmaceutically acceptable carrier, methods of using the compositions to inhibit expression of a target cFLIP gene, and methods of using the pharmaceutical compositions to treat cancer, particularly TRAIL-resistant cancer cells.

I. Definitions

For convenience, the meaning of certain terms and phrases used in the specification, examples, and appended claims, are provided below.

As used herein, “target gene” refers to a section of a DNA strand of a double-stranded DNA that is complementary to a section of a DNA strand, including all transcribed regions, that serves as a matrix for transcription. A target gene, usually the sense strand, is a gene whose expression is to be selectively inhibited or silenced through RNA interference. As used herein, the term “target gene” specifically encompasses any cellular gene or gene fragment whose expression or activity is associated with an inhibition or blocking effect on cell apoptosis, including genes or gene fragments whose expression or activity is implicated in resistance to an apoptosis-inducing substance, such as TRAIL. The term “apoptosis” (also known as “programmed cell death”) is recognized in the art as an active process of gene-directed cellular self-destruction.

As used herein, the terms “cellular FLICE-like inhibitory protein gene” and “cFLIP gene” refer to a DNA sequence encoding a gene product or a gene product fragment that inhibits cell apoptosis and includes at least one death effector domain. A “gene product” or a “gene product fragment” is defined as an encoded protein or encoded protein fragment. As used herein, the term “gene product” includes a primary transcript, such as mRNA. The term “cFLIP gene” includes all derivatives (natural and synthetic) or alleles of the DNA sequence, provided they are functionally homologous to the natural sequence. All naturally occurring DNA sequences exhibiting the same functions, but showing characteristic mutations of the natural DNA sequence due to evolutionary development, qualify as alleles. Artificial alterations of the natural DNA sequence can be introduced by known methods. The mutations can be introduced at a certain DNA sequence site by synthesizing the appropriate oligonucleotides including a certain mutation sequence.

As used herein, the terms “apoptosis-inducing drug,” “bioactive substance,” and “bioactive substance that induces receptor-mediated apoptosis” are used interchangeably to refer to any substance which induces the death of cancer cells, but not normal cells. The term “TNF-related ligand” refers to any ligand that binds to a receptor for the tumor necrosis factor type. The term “TNF-related ligand” includes ligands that bind to the receptors TRAMP, CD95, and TNFR-1, as well as the receptor for the death-inducing ligand TRAIL. As used herein, the term “TRAIL” refers to the tumor necrosis factor-related apoptosis-inducing ligand (also called “Apo2L”), which is described, for example, in Wiley, et al., Immunity (1995) 3:673-683, which is incorporated by reference herein.

The term “TRAIL-resistant cancer cells” refers to cancer cells that are not killed by TRAIL. Cells are considered to be “TRAIL-sensitive” when less than 50% of the cells is a cell population which has been exposed to TRAIL die following exposure. Cells are considered to be “TRAIL-resistant” when 50% or more of the cells in the cell population survive exposure to TRAIL.

The term “complementary RNA strand” (also referred to herein as the “antisense strand”) refers to the strand of a dsRNA which is complementary to an mRNA transcript that is formed during expression of the target gene, or its processing products. As used herein, the term “complementary nucleotide sequence” refers to the region on the complementary RNA strand that is complementary to an mRNA transcript of a portion of the target gene. “dsRNA” refers to a ribonucleic acid molecule having a duplex structure comprising two complementary and anti-parallel nucleic acid strands. Not all nucleotides of a dsRNA must exhibit Watson-Crick base pairs; the two RNA strands may be substantially complementary (i.e., having no more than one or two nucleotide mismatches). The maximum number of base pairs is the number of nucleotides in the shortest strand of the dsRNA. The RNA strands may have the same or a different number of nucleotides. The dsRNA is less than 30, preferably less than 25, more preferably 21 to 24, and most preferably 23 nucleotides in length. dsRNAs of this length are particularly efficient in inhibiting the expression of the target cFLIP gene. “Introducing into” means uptake or absorption in the cell, as is understood by those skilled in the art. Absorption or uptake of dsRNA can occur through cellular processes, or by auxiliary agents or devices, including vector delivery. For example, for in vivo delivery, dsRNA can be injected into a tissue site or administered systemically. In vitro delivery includes methods known in the art such as electroporation and lipofection.

As used herein, a “nucleotide overhang” refers to the unpaired nucleotide or nucleotides that protrude from the duplex structure when a 3′-end of one RNA strand extends beyond the 5′-end of the other strand, or vice versa.

As used herein and as known in the art, the term “identity” is the relationship between two or more polynucleotide sequences, as determined by comparing the sequences. Identity also means the degree of sequence relatedness between polynucleotide sequences, as determined by the match between strings of such sequences. Identity can be readily calculated (see, e.g., Computation Molecular Biology, Lesk, A. M., eds., Oxford University Press, New York (1998), and Biocomputing: Informatics and Genome Projects, Smith, D. W., ed., Academic Press, New York (1993), both of which are incorporated by reference herein). While there exist a number of methods to measure identity between two polynucleotide sequences, the term is well known to skilled artisans (see, e.g., Sequence Analysis in Molecular Biology, von Heinje, G., Academic Press (1987); and Sequence Analysis Primer, Gribskov., M. and Devereux, J., eds., M. Stockton Press, New York (1991)). Methods commonly employed to determine identity between sequences include, for example, those disclosed in Carillo, H., and Lipman, D., SIAM J. Applied Math. (1988) 48:1073. “Substantially identical,” as used herein, means there is a very high degree of homology (preferably 100% sequence identity) between the sense strand of the dsRNA and the corresponding part of the target gene. However, dsRNA having greater than 90%, or 95% sequence identity may be used in the present invention, and thus sequence variations that might be expected due to genetic mutation, strain polymorphism, or evolutionary divergence can be tolerated. Although 100% identity is preferred, the dsRNA may contain single or multiple base-pair random mismatches between the RNA and the target gene.

As used herein, the term “treatment” refers to the application or administration of a therapeutic agent to a patient, or application or administration of a therapeutic agent to an isolated tissue or cell line from a patient, who has a disorder, e.g., a disease or condition, a symptom of disease, or a predisposition toward a disease, with the purpose to cure, heal, alleviate, relieve, alter, remedy, ameliorate, improve, or affect the disease, the symptoms of disease, or the predisposition toward disease.

As used herein, a “pharmaceutical composition” comprises a pharmacologically effective amount of a dsRNA and a pharmaceutically acceptable carrier. As used herein, “pharmacologically effective amount,” “therapeutically effective amount” or simply “effective amount” refers to that amount of an RNA effective to produce the intended pharmacological, therapeutic or preventive result. For example, if a given clinical treatment is considered effective when there is at least a 25% reduction in a measurable parameter associated with a disease or disorder, a therapeutically effective amount of a drug for the treatment of that disease or disorder is the amount necessary to effect at least a 25% reduction in that parameter.

The term “pharmaceutically acceptable carrier” refers to a carrier for administration of a therapeutic agent. Such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

As used herein, a “transformed cell” is a cell into which a dsRNA molecule has been introduced by means of recombinant DNA techniques.

II. Double-Stranded Ribonucleic Acid (dsRNA)

In one embodiment, the invention relates to a double-stranded ribonucleic acid (dsRNA) having a nucleotide sequence which is substantially identical to at least a portion of a cFLIP gene. The dsRNA comprises two RNA strands that are sufficiently complementary to hybridize to form the duplex structure. One strand of the dsRNA comprises the nucleotide sequence that is substantially identical to a portion of the target gene (the “sense” strand), and the other strand (the “complementary” or “antisense” strand) comprises a sequence that is complementary to an RNA transcript of the target cFLIP gene. The complementary region is less than 25 nucleotides, preferably 19 to 24 nucleotides, more preferably 20 to 24 nucleotides, even more preferably 21 to 23 nucleotides, and most preferably 22 or 23 nucleotides in length. The dsRNA is less than 30 nucleotides, preferably less than 25 nucleotides, and most preferably between 21 and 24 nucleotides in length. The dsRNA can be synthesized by standard methods known in the art, e.g., by use of an automated DNA synthesizer, such as are commercially available from Biosearch, Applied Biosystems, Inc. In specific embodiments, the complementary (antisense) RNA strand of the dsRNA comprises the sequence set forth in SEQ ID NO:2 and the second (sense) RNA strand comprises the sequence set forth in SEQ ID NO:1; or the complementary (antisense) RNA strand of the dsRNA comprises the sequence set forth in SEQ ID NO:4 and the second (sense) RNA strand comprises the sequence set forth in SEQ ID NO:3; or the complementary (antisense) RNA strand of the dsRNA comprises the sequence set forth in SEQ ID NO:7 and the second (sense) RNA strand comprises the sequence set forth in SEQ ID NO:1; or the complementary (antisense) RNA strand of the dsRNA comprises the sequence set forth in SEQ ID NO:8 and the second (sense) RNA strand comprises the sequence set forth in SEQ ID NO:3.

In one embodiment, at least one end of the dsRNA has a single-stranded nucleotide overhang of 1 to 4, preferably 2 or 3 nucleotides. dsRNAs having at least one nucleotide overhang have unexpectedly superior inhibitory properties than their blunt-ended counterparts. Moreover, the present inventors have discovered that the presence of only one nucleotide overhang strengthens the interference activity of the dsRNA, without effecting its overall stability. dsRNA having only one overhang has proven particularly stable and effective in vivo, as well as in a variety of cells, cell culture mediums, blood, and serum. Preferably, the single-stranded overhang is located at the 3′-terminal end of the complementary (antisense) RNA strand or, alternatively, at the 3′-terminal end of the second (sense) strand. The dsRNA may also have a blunt end, preferably located at the 5′-end of the complementary (antisense) strand. Such dsRNAs have improved stability and inhibitory activity, thus allowing administration at low dosages, i.e., less than 5 milligrams per kilogram body weight of the recipient per day. Preferably, the dsRNA has a nucleotide overhang at the 3′-end of the complementary strand, and the blunt end of the dsRNA is at the 5′-end of the complementary RNA strand. In a particularly preferred embodiment, the complementary RNA strand is 23 nucleotides in length, the sense RNA strand is 21 nucleotides in length, wherein the dsRNA has a 1 or 2 nucleotide overhang at one end and is blunt-ended at the other end, and wherein the nucleotide overhang is at the 3′-end of the complementary RNA strand and the blunt end is at the 5′-end of the complementary RNA strand.

III. Pharmaceutical Compositions Comprising dsRNA

In one embodiment, the invention relates to a pharmaceutical composition comprising a dsRNA, as described in the preceding section, and a pharmaceutically acceptable carrier, as described below. The pharmaceutical composition comprising the dsRNA is useful for inhibiting the expression or activity of a cFLIP gene, for improving the effectiveness of an apoptosis-inducing drug, and for treating cancer.

The pharmaceutical compositions of the present invention are administered in dosages sufficient to inhibit expression of the target gene, cFLIP. The present inventors have found that, because of their efficiency, compositions comprising the dsRNA of the invention can be administered at surprisingly low dosages. A maximum dosage of 5 milligrams (mg) dsRNA per kilogram (kg) body weight of recipient per day is sufficient to inhibit or completely suppress expression of the target gene.

In general, a suitable dose of dsRNA will be in the range of 0.01 to 5.0 milligrams per kilogram body weight of the recipient per day, preferably in the range of 0.1 to 2.5 milligrams per kilogram body weight per day, more preferably in the range of 0.1 to 100 micrograms per kilogram body weight per day, more preferably in the range of 0.1 to 200 micrograms per kilogram body weight per day, even more preferably in the range of 0.1 to 50 micrograms per kilogram body weight per day, and most preferably in the range of 0.1 to 25 micrograms per kilogram body weight per day. The pharmaceutical composition may be administered once daily, or the dsRNA may be administered as two, three, four, five, six or more sub-doses at appropriate intervals throughout the day. In that case, the dsRNA contained in each sub-dose must be correspondingly smaller in order to achieve the total daily dosage. The dosage unit can also be compounded for delivery over several days, e.g., using a conventional sustained release formulation which provides sustained release of the dsRNA over a several day period. Sustained release formulations are well known in the art. In this embodiment, the dosage unit contains a corresponding multiple of the daily dose.

The skilled artisan will appreciate that certain factors may influence the dosage and timing required to effectively treat a subject, including but not limited to the severity and type of cancer, previous treatments, the percentage of TRAIL-resistant cancer cells and their degree of resistance, the general health and/or age of the subject, and other diseases present. Moreover, treatment of a subject with a therapeutically effective amount of a composition can include a single treatment or a series of treatments. Estimates of effective dosages and in vivo half-lives for the individual dsRNAs encompassed by the invention can be made using conventional methodologies or on the basis of in vivo testing using an appropriate animal model, as described elsewhere herein.

Advances in mouse genetics have generated a number of mouse models for the study of various human diseases. For example, mouse models are available for nonobese diabetes (“NOD”) (Kishimoto, H. and J. Sprent, Nat. Immunol. (2001), 2(11):1025-31); perforin-deficient mice (Taylor, M. A., et al., J. Immunol. (2001), 167(8):4230-37 and Taylor, M. A., et al., J. Immunol. (2001), 167(8):7207); and experimental autoimmune encephalomyelitis (“EAE”) (Djerbi, M., et al., J. Immunol. (2003), 170(4):2064-73). Such models are useful for studying the effect of cFLIP overexpression on apoptosis, and thus are useful for in vivo testing of the dsRNAs of the present inventing as well as for determining a therapeutically effective dose. Mouse models are also available for hematopoietic malignancies such as leukemias, lymphomas and acute myelogenous leukemia. The MMHCC (Mouse models of Human Cancer Consortium) web page (emice.nci.nih.gov), sponsored by the National Cancer Institute, provides disease-site-specific compendium of known cancer models, and has links to the searchable Cancer Models Database (cancermodels.nci.nih.gov ), as well as the NCI-MMHCC mouse repository. Examples of the genetic tools that are currently available for the modeling of leukemia and lymphomas in mice, are described in the following references: Maru, Y., Int. J. Hematol. (2001) 73:308-322; Pandolfi, P. P., Oncogene (2001) 20:5726-5735; Pollock, J. L., et al., Curr. Opin. Hematol. (2001) 8:206-211; Rego, E. M., et al., Semin. in Hemat. (2001) 38:4-70; Shannon, K. M., et al. “Modeling myeloid leukemia tumors suppressor gene inactivation in the mouse” , Semin. Cancer Biol., (2001), 11:191-200; Van Etten, R. A., Curr. Opin. Hematol., (2001), 8:224-230; Wong, S., et al., Oncogene, (2001), 20:5644-5659; Phillips, J. A., Cancer Res. (2000) 52(2):437-43; Harris, A. W., et al, J. Exp. Med. (1988) 167(2):353-7-1-; Zeng, X X et al., Blood. (1988) 92(10):3529-36; Eriksson, B., et al., Exp. Hematol. (1999) 27(4):682-8; and Kovalchuk, A.,. et al., J. Exp. Med.(2000) 192(8):1183-90. Mouse repositories can also be found at: The Jackson Laboratory, Charles River Laboratories, Taconic, Harlan, Mutant Mouse Regional Resource Centers (MMRRC) National Network and at the European Mouse Mutant Archive.

The pharmaceutical compositions encompassed by the invention may be administered by any means known in the art including, but not limited to oral or parenteral routes, including intravenous, intramuscular, intraparenteral, subcutaneous, transdermal, airway (aerosol), rectal, vaginal and topical (including buccal and sublingual) administration. In preferred embodiments, the pharmaceutical compositions are administered by intravenous or intraparenteral infusion or injection.

For oral administration, the dsRNAs useful in the invention will generally be provided in the form of tablets or capsules, as a powder or granules, or as an aqueous solution or suspension.

Tablets for oral use may include the active ingredients mixed with pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

Capsules for oral use include hard gelatin capsules in which the active ingredient is mixed with a solid diluent, and soft gelatin capsules wherein the active ingredients is mixed with water or an oil such as peanut oil, liquid paraffin or olive oil.

For intramuscular, intraparenteral, subcutaneous and intravenous use, the pharmaceutical compositions of the invention will generally be provided in sterile aqueous solutions or suspensions, buffered to an appropriate pH and isotonicity. Suitable aqueous vehicles include Ringer's solution and isotonic sodium chloride. In a preferred embodiment, the carrier consists exclusively of an aqueous buffer. In this context, “exclusively” means no auxiliary agents or encapsulating substances are present which might affect or mediate uptake of dsRNA in the cells that express the target gene. Such substances include, for example, micellar structures, such as liposomes or capsids, as described below. Surprisingly, the present inventors have discovered that compositions containing only naked dsRNA and a physiologically acceptable solvent are taken up by cells, where the dsRNA effectively inhibits expression of the target gene. Although microinjection, lipofection, viruses, viroids, capsids, capsoids, or other auxiliary agents are required to introduce dsRNA into cell cultures, surprisingly these methods and agents are not necessary for uptake of dsRNA in vivo. Aqueous suspensions according to the invention may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

The pharmaceutical compositions useful according to the invention also include encapsulated formulations to protect the dsRNA against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811; PCT publication WO 91/06309; and European patent publication EP-A-43075, which are incorporated by reference herein.

In one embodiment, the encapsulated formulation comprises a viral coat protein. In this embodiment, the dsRNA may be bound to, associated with, or enclosed by at least one viral coat protein. The viral coat protein may be derived from or associated with a virus, such as a polyoma virus, or it may be partially or entirely artificial. For example, the coat protein may be a Virus Protein 1 and/or Virus Protein 2 of the polyoma virus, or a derivative thereof.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred.

The data obtained from cell culture assays and animal studies can be used in formulation a range of dosage for use in humans. The dosage of compositions of the invention lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range of the compound or, when appropriate, of the polypeptide product of a target sequence (e.g., achieving a decreased concentration of the polypeptide) that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In addition to their administration individually or as a plurality, as discussed above, the dsRNAs useful according to the invention can be administered in combination with other known agents effective in treatment of diseases. In any event, the administering physician can adjust the amount and timing of dsRNA administration on the basis of results observed using standard measures of efficacy known in the art or described herein.

In one embodiment, the pharmaceutical composition comprises, in addition to the dsRNA of the invention, an apoptosis-inducing drug. The apoptosis-inducing drug (or bioactive substance) induces receptor-mediated apoptosis which induces the death of cancer cells. The apoptosis-inducing drug can be a tumor necrosis factor (TNF) or TNF-related ligand, including, without limitation, ligands that bind to the receptors TRAMP, CD95, and TNFR-1, as well as the receptor for the death-inducing ligand TRAIL. In a preferred embodiment, the apoptosis-inducing drug is the tumor necrosis factor-related apoptosis-inducing ligand (TRAIL).

For oral administration, the dsRNAs useful in the invention will generally be provided in the form of tablets or capsules, as a powder or granules, or as an aqueous solution or suspension.

IV. Methods for Inhibiting Expression of a cFLIP Gene

In another aspect, the invention relates to a method for inhibiting the expression of a cFLIP gene in a mammal. The method comprises administering a composition of the invention to the mammal such that expression of the target cFLIP gene is silenced. Because of their high specificity, the dsRNAs of the present invention specifically target RNAs (primary or processed) of target cFLIP genes, and at surprisingly low dosages. Compositions and methods for inhibiting the expression of these target genes using dsRNAs can be performed as described elsewhere herein.

In one embodiment, the invention comprises administering a composition comprising a dsRNA, wherein the dsRNA comprises a nucleotide sequence which is complementary to at least a part of an RNA transcript of the target cFLIP gene of a mammal (e.g., human). The composition may be administered by any means known in the art including, but not limited to oral or parenteral routes, including intravenous, intramuscular, intraparenteral, subcutaneous, transdermal, airway (aerosol), rectal, vaginal and topical (including buccal and sublingual) administration. In preferred embodiments, the compositions are administered by intravenous or intraparenteral infusion or injection.

V. Methods for Improving the Effectiveness of an Apoptosis-Inducing Drug

To increase the efficacy of treatment with a receptor-mediated apoptotic drug, such as TRAIL, the cancer cells may be treated with a dsRNA of the present invention. The method comprises administering a pharmaceutical composition, as discussed above, to the mammal such that expression of the target cFLIP gene is silenced. Because of their high specificity, the dsRNAs of the present invention specifically target RNAs (primary or processed) of target cFLIP genes, and at surprisingly low dosages. Compositions and methods for inhibiting the expression of these target genes using dsRNAs can be performed as described elsewhere herein. The treatment may be used in combination with other means of treatment such as surgery, chemotherapy, or radiation therapy.

Preferably, the treatment comprises intravenous administration of the pharmaceutical composition of the present invention. In another aspect, the present invention comprises a method for increasing the efficacy of an apoptosis-inducing drug, such as TRAIL. The method comprises treating cancer cells with a pharmaceutical composition comprising an effective amount of TRAIL and an effective amount of dsRNA sufficient to induce apoptosis in at least a portion of the treated cancer cells.

In an embodiment, the cancer cells are treated with the dsRNA of the present invention prior to being treated with TRAIL. Alternatively, the cancer cells may be treated with dsRNA and TRAIL concurrently.

Preferably, the dose of TRAIL in the pharmaceutical composition results in a local concentration of TRAIL at the tumor which ranges from 1 to 1,000 nanograms (ng) per millimeter (mg). More preferably, the dose of TRAIL in the pharmaceutical composition results in a local concentration of TRAIL at the tumor which ranges from 200 to 600 ng/ml. Even more preferably, the dose of TRAIL in the pharmaceutical composition results in a local concentration of TRAIL at the tumor which ranges from 350 to 450 ng/ml.

The cancer cells treatment using the compositions and methods of the present invention include cancer cells which have shown some sensitivity to TRAIL, as well as cancer cells with are at least partially resistant to TRAIL or potentially TRAIL-resistant. Such cells may include, for example, breast cancer cells, kidney cancer cells, lung cancer cells, colon cancer cells, prostate cancer cells, and glioblastomas.

In an embodiment, treatment of cells with TRAIL and dsRNA is associated with an increase in the amount of activated caspase enzymes in at least a portion of the treated cells. Preferably, the caspases which are activated upon exposure of cells to TRAIL and dsRNA comprise caspase-8, caspase-3, caspase-9, or caspase-7.

VI. Methods for Treating Cancer

In another aspect, the present invention comprises a method for inducing cell death in cancer cells, the method comprising treating cancer cells with an effective amount of an apopotisis-inducing drug, such as TRAIL, and an effective amount of a dsRNA of the present invention sufficient to induce apoptosis in at least a portion of the treated cancer cells. In a preferred embodiment, the dsRNA is less than 25 nucleotides in length, and comprises a 2 or 3 nucleotide overhang on a 3′-terminus of the complementary RNA strand. In an embodiment, the cancer cells are treated with the dsRNA prior to being treated with the apoptosis inducing drug. Alternatively, the cancer cells may be treated with the dsRNA and apoptosis inducing drug substantially concurrently.

The method for treating cancer comprises administering a pharmaceutical composition, as discussed above, to the mammal such that expression of the target cFLIP gene is silenced and apoptosis is induced. Because of their high specificity, the dsRNAs of the present invention specifically target RNAs (primary or processed) of target cFLIP genes, and at surprisingly low dosages. Compositions and methods for treating cancers using dsRNA and an apoptotis drug can be performed as described above. The treatment may be used in combination with other means of treatment such as surgery, chemotherapy, or radiation therapy.

Preferably, the treatment comprises intravenous administration of the pharmaceutical composition of the present invention. In another aspect, the present invention comprises a method for treating cancer cells with a pharmaceutical composition comprising an effective amount of TRAIL and an effective amount of dsRNA sufficient to induce apoptosis in at least a portion of the treated cancer cells.

In an embodiment, the cancer cells are treated with the dsRNA of the present invention prior to being treated with the apoptotic-inducing drug. Alternatively, the cancer cells may be treated with dsRNA and apoptosis-inducing drug concurrently.

Preferably, the dose of apoptosis-inducing drug (e.g., TRAIL) in the pharmaceutical composition results in a local concentration of the drug in the tumor which ranges from 1 to 1,000 nanograms (ng) per millimeter (mg). More preferably, the dose of apoptosis-inducing drug in the pharmaceutical composition results in a local concentration of the drug at the tumor which ranges from 200 to 600 ng/ml. Even more preferably, the dose of apoptosis-inducing drug in the pharmaceutical composition results in a local concentration of drug at the tumor which ranges from 300 to 500 ng/ml.

The cancer cells treatable using the compositions and methods of the present invention include cancer cells which have shown some sensitivity to TRAIL, as well as cancer cells with are at least partially resistant to TRAIL or potentially TRAIL-resistant. Such cells may include, for example, breast cancer cells, kidney cancer cells, colon cancer cells, prostate cancer cells, and glioblastomas.

In one embodiment, treatment of cancer cells with an apoptosis-inducing drug and dsRNA is associated with an increase in the amount of activated caspase enzymes in at least a portion of the treated cells. Preferably, the caspases which are activated upon exposure of cells to the apoptosis-inducing drug and dsRNA comprise caspase-8, caspase-3, caspase-9, or caspase-7.

In one embodiment, the dsRNA can act as novel therapeutic agents for controlling one or more of cellular proliferative and/or differentiative disorders. The method comprises administering a pharmaceutical composition of the invention to the patient (e.g., human), such that expression of the target gene is silenced. Because of their high specificity, the dsRNAs of the present invention specifically target mRNAs of target genes of diseased cells and tissues, as described below, and at surprisingly low dosages.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of pancreas, prostate, brain, kidney, colon, lung, breast and liver origin. As used herein, the terms “cancer,” “hyperproliferative,” and “neoplastic” refer to cells having the capacity for autonomous growth, i.e., an abnormal state of condition characterized by rapidly proliferating cell growth. These terms are meant to include all types of cancerous growths or oncogenic processes, metastatic tissues or malignantly transformed cells, tissues, or organs, irrespective of histopathologic type or stage of invasiveness. Proliferative disorders also include hematopoietic neoplastic disorders, including diseases involving hyperplastic/neoplatic cells of hematopoietic origin, e.g., arising from myeloid, lymphoid or erythroid lineages, or precursor cells thereof.

The pharmaceutical compositions encompassed by the invention may be administered by any means known in the art including, but not limited to oral or parenteral routes, including intravenous, intramuscular, intraparenteral, subcutaneous, transdermal, airway (aerosol), rectal, vaginal and topical (including buccal and sublingual) administration. In preferred embodiments, the pharmaceutical compositions are administered by intravenous or intraparenteral infusion or injection.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Example 1

### Inhibition of cFLIP Gene Expression by RNA Interference

Synthesis and Preparation of dsRNAs

Oligoribonucleotides are synthesized with an RNA synthesizer (Expedite 8909, Applied Biosystems, Weiterstadt, Germany) and purified by High Pressure Liquid Chromatography (HPLC) using NucleoPac PA-100 columns, 9×250 mm (Dionex Corp.; low salt buffer: 20 mM Tris, 10 mM NaClO₄, pH 6.8, 10% acetonitrile; the high-salt buffer was: 20 mM Tris, 400 mM NaClO4, pH 6.8, 10% acetonitrile. flow rate: 3 ml/min). Formation of double stranded siRNAs is then achieved by heating a stoichiometric mixture of the individual complementary strands (10 M) in 10 mM sodium phosphate buffer, pH 6.8, 100 mM NaCl, to 80-90° C., with subsequent slow cooling to room temperature over 6 hours,

In addition, dsRNA molecules with linkers may be produced by solid phase synthesis and addition of hexaethylene glycol as a non-nucleotide linker (D. Jeremy Williams, Kathleen B. Hall, Biochemistry (1996) 35:14665-14670). A hexaethylene glycol linker phosphoramidite (Chruachem Ltd, Todd Campus, West of Scotland Science Park, Acre Road, Glasgow, G20 OUA, Scotland, UK) is coupled to the support bound oligoribonucleotide employing the same synthetic cycle as for standard nucleoside phosphoramidites (Proligo Biochemie GmbH, Georg-Hyken-Str. 14, Hamburg, Germany) but with prolonged coupling times. Incorporation of linker phosphoramidite is comparable to the incorporation of nucleoside phosphoramidites.

The double-stranded oligoribonucleotides having the following sequences were synthesized (SEQ ID NO: 1 to SEQ ID NO: 9 in the sequence protocol):

dsRNA-F1, corresponding to nucleotides 472 to 492 of the cFLIP-L gene:

| | | |
|---|---|---|
| S2: | 5′-GUGCCGGGAUGUUGCUAUAGA-3′ | (SEQ ID NO: 1) |
| S1: | 3′-AACACGGCCCUACAACGAUAU-5′ | (SEQ ID NO: 2) |

dsRNA-F2, corresponding to nucleotides 908 to 928 of the cFLIP-L gene:

| | | |
|---|---|---|
| S2: | 5′-CAAGGAGCAGGGACAAGUUAC-3′ | (SEQ ID NO: 3) |
| S1: | 3′-AAGUUCCUCGUCCCUGUUCAA-5′ | (SEQ ID NO: 4) |

dsRNA-neo, which is complementary to a sequence of the neomycin resistance gene:

| | | |
|---|---|---|
| S2: | 5′-GAUGAGGAUCGUUUCGCAUGA-3′ | (SEQ ID NO: 5) |
| S1: | 3′-UCCUACUCCUAGCAAAGCGUA-5′ | (SEQ ID NO: 6) |

dsRNA-F3, corresponding to nucleotides 472 to 494 of the cFLIP-L gene:

| | | |
|---|---|---|
| S2: | 5′-GUGCCGGGAUGUUGCUAUAGA-3′ | (SEQ ID NO: 1) |
| S1: | 3′-AACACGGCCCUACAACGAUAUCU-5′ | (SEQ ID NO: 7) |

dsRNA-F4, corresponding to nucleotides 908 to 930 of the cFLIP-L gene:

| | | |
|---|---|---|
| S2: | 5′-CAAGGAGCAGGGACAAGUUAC-3′ | (SEQ ID NO: 3) |
| S1: | 3′-AAGUUCCUCGUCCCUGUUCAAUG-5′ | (SEQ ID NO: 8) |

dsRNA control, which is complementary to a sequence of the neomycin resistance gene:

| | | |
|---|---|---|
| S2: | 5′-GAUGAGGAUCGUUUCGCAUGA-3′ | (SEQ ID NO: 5) |
| S1: | 3′-UCCUACUCCUAGCAAAGCGUACU-5′ | (SEQ ID NO: 9) |

KB cells were obtained from the American Type Culture Collection (ATCC), Deposit No. ATCC No. CCL-17. SV80 cells were obtained from CLS Corp., 69123 Heidelberg, Germany, Order No. 0345 HU (ATCC No.: Crl-7725).

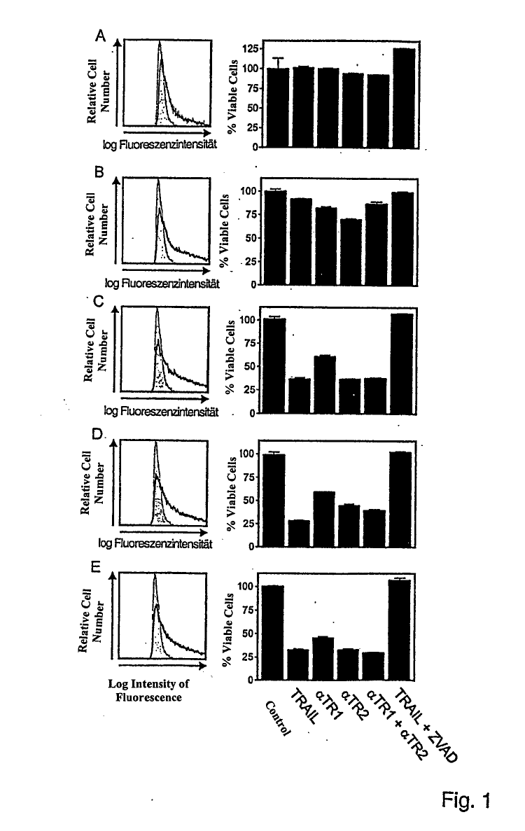

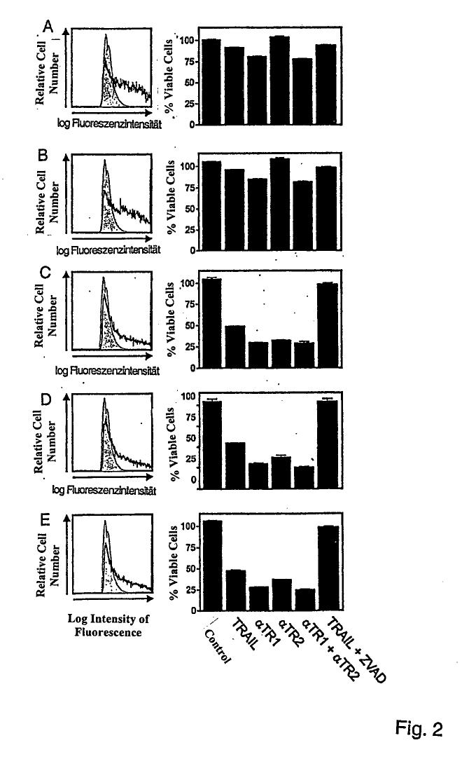

In each case, 10⁷ SV80 cells and KB cells per ml are transfected twice by means of electroporation on successive days without (FIG. 1A, FIG. 2A), or with 150 nmol/l dsRNA-neo (FIG. 1B, FIG. 2B), 150 nmol/l dsRNA-F1 (FIG. 1C, FIG. 2C), 150 nmol/l dsRNA-F2 (FIG. 1D, FIG. 2D), or with a mixture of 75 nmol/l each of dsRNA-F1 and dsRNA-F2 (FIG. 1E, FIG. 2E). A GFP (green fluorescent protein) expression plasmid was added to the electroporation solution of each of the first electroporations to evaluate the efficiency of each transfection. The cells were harvested one day after the first electroporation. Transfection efficiency was determined on a portion of the cells by measuring fluorescence intensity by flow cytometry.

The fluorescence intensity of these cells is represented by a thick line in the left field of FIGS. 1A-E and 2A-E. The thin lines in the same field represents the fluorescence intensity of the same cells without the GFP expression plasmid. To maximize transfection efficiency, another portion of the cells were electroporated a second time with the same dsRNA as used on the first day. The cells were then seeded in 100 microliter (μl) medium in wells in 96-well plates. The cells were incubated the following day for 9 hours with:

Flag-coupled soluble TRAIL (“TRAIL”) cross-linked with the monoclonal M2 anti-flag antibody, which can stimulate both TRAIL-R1 and TRAIL-R2,

Specific rabbit antiserum (1:500) that is agonistic to TRAIL-R1 (“αTR1”) and/or TRAIL-R2 (“αTR2”), or

Flag-coupled, cross-linked soluble TRAIL, as above, in the presence of 20 μmol/l of the caspase inhibitor z-VAD-fmk (“TRAIL+ZVAD”).

Finally, the proportion of living cells was determined by means of crystal violet staining.

From the results shown in FIGS. 1 and 2, it is clear that transfection efficiency of the GFP expression plasmid was not affected by dsRNA in any of the cell lines tested. DsRNA-F1 and dsRNA-F2 (FIGS. 1C-E, FIGS. 2C-E) had a significant sensitizing effect on TRAIL-R1- and TRAIL-R2-mediated apoptosis in KB cells and SV80 cells. However, this was not the case with dsRNA-neo (FIG. 1B, FIG. 2B) or with electroporation without dsRNA (mock electroporation) (FIG. 1A, FIG. 2A). ZVAD eliminated the sensitization to TRAIL-induced apoptosis. This points to the involvement of caspases (FIGS. 1C-E, FIGS. 2C-E).

In a further experiment, KB cells were also sensitized to FasL- and TNF-induced apoptosis by treatment with dsRNA-F1 and dsRNA-F2 (data not shown).

The exemplified dsRNAs (dsRNA-F1, dsRNA-F2, and dsRNA-neo) each comprise a 2-nucleotide overhang at both ends. The overhangs were formed from the 3′-ends of the S1 and S2 strands. The double-stranded region was 19 nucleotides pairs long. Additional experiments on the inhibition of FLIP-mRNA expression were carried out with the dsRNAs dsRNA-F3, dsRNA-F4, and control dsRNA. In these, each of the dsRNA comprised a single 2-nucleotide overhang at the 3′-end of the S1 strand. The double-stranded region was 21 nucleotides in length. Quantitative analysis revealed that dsRNA-F3 and dsRNA-F4 are approximately as effective in inhibiting the expression of a GFP-FLIP fusion protein as are dsRNA-F1 and dsRNA-F2.

### Example 2

### Treatment of a Cancer Patient with cFLIP dsRNAs

In this Example, cFLIP dsRNAs are injected into a pancreatic cancer patient and shown to specifically inhibit cFLIP gene expression.

dsRNA Administration and Dosage

The present example provides for pharmaceutical compositions for the treatment of human cancer patients comprising a therapeutically effective amount of a cFLIP dsRNA as described herein, in combination with a pharmaceutically acceptable carrier or excipient. cFLIP dsRNAs according to the invention may be formulated as described above (e.g., oral or parenteral administration). The pharmaceutical compositions may be administered in any effective, convenient manner including, for instance, administration by topical, oral, anal, vaginal, intravenous, intraparenteral, intramuscular, subcutaneous, intranasal or intradermal routes among others. One of skill in the art can readily prepare dsRNAs for injection using such carriers that include, but are not limited to, saline, buffered saline, dextrose, water, glycerol, ethanol, and combinations thereof. Additional examples of suitable carriers are found in standard pharmaceutical texts, e.g. Remington's Pharmaceutical Sciences, 16th edition, Mack Publishing Company, Easton, Pa., 1980.

The dosage of the cFLIP dsRNAs will vary depending on the form of administration. The dose of apoptosis-inducing drug (e.g., TRAIL) in the pharmaceutical composition results in a local concentration of the drug in the tumor which ranges from 1 to 1,000 nanograms (ng) per millimeter (mg), preferably 200 to 600 ng/ml. Even more preferably, the dose of apoptosis-inducing drug in the pharmaceutical composition results in a local concentration of apoptosis-inducing drug at the tumor which ranges from 300 to 500 ng/ml.

In addition to the active ingredients, the compositions usually also contain suitable buffers, for example phosphate buffer, to maintain an appropriate pH and sodium chloride, glucose or mannitol to make the solution isotonic. The administering physician will determine the daily dosage which will be most suitable for an individual and will vary with the age, gender, weight and response of the particular individual, as well as the severity of the patient's symptoms. The above dosages are exemplary of the average case. There can, of course, be individual instances where higher or lower dosage ranges are merited, and such are within the scope of this invention. The cFLIP dsRNAs of the present invention and the apoptosis-inducing drug (e.g., TRAIL) may be administered alone or with additional dsRNA species or in combination with other pharmaceuticals.

RNA Purification and Analysis

Efficacy of the cFLIP dsRNA treatment is determined at defined intervals after the initiation of treatment using real time PCR on total RNA extracted from tissue biopsies. Cytoplasmic RNA from tissue biopsies, taken prior to and during treatment, may be purified using RNeasy Kit (Qiagen, Hilden) and cFLIP mRNA levels may be quantitated by real time RT-PCR as described previously (Eder, M., et al., Leukemia (1999) 13:1383-1389; and Scherr, M et al., BioTechniques (2001) 31:520-526). Analysis of cFLIP mRNA levels before and during treatment by real time PCR, provides the attending physician with a rapid and accurate assessment of treatment efficacy as well as the opportunity to modify the treatment regimen in response to the patient's symptoms and disease progression.

### Example 3

cFLIP dsRNA Expression Vectors

In another aspect of the invention, cFLIP-specific dsRNA molecules that interact with cFLIP target RNA molecules, and which modulate cFLIP gene expression activity, are expressed from transcription units inserted into DNA or RNA vectors (see, for example, Couture, et al, TIG (1996) 12:5-10; Skillern, et al., International PCT Publication No. WO 00/22113; Conrad, International PCT Publication No. WO 00/22114, and Conrad, U.S. Pat. No. 6,054,299). These transgenes can be introduced as a linear construct, a circular plasmid, or a viral vector, which can be incorporated and inherited as a transgene integrated into the host genome. The transgene can also be constructed to permit integration as an extrachromosomal plasmid (Gassmann, et al., Proc. Natl. Acad. Sci. USA (1995) 92:1292).

The individual strands of a cFLIP dsRNA can be transcribed by promoters on two separate expression vectors and co-transfected into a target cell. Alternatively each individual strand of the dsRNA can be transcribed by promoters both of which are located on the same expression plasmid. In a preferred embodiment, a dsRNA is expressed as an inverted repeat joined by a linker polynucleotide sequence such that the dsRNA has a stem and loop structure.

The recombinant cFLIP dsRNA expression vectors are preferably DNA plasmids or viral vectors. cFLIP dsRNA expressing viral vectors can be constructed using, for example, adeno-associated virus (for a review, see Muzyczka, et al., Curr. Topics Micro. Immunol. (1992) 158:97-129), adenovirus (see, for example, Berkner, et al., BioTechniques (1988) 6:616; Rosenfeld, et al., Science (1991) 252:431-434, and Rosenfeld, et al., Cell (1992) 68:143-155), or alphavirus as well as others known in the art. Retroviruses have been used to introduce a variety of genes into many different cell types, including epithelial cells, in vitro and/or in vivo (see, for example, Eglitis, et al., Science (1985) 230:1395-1398; Danos and Mulligan, Proc. NatI. Acad. Sci. USA (1988) 85:6460-6464; Wilson, et al., Proc. NatI. Acad. Sci. USA (1988) 85:3014-3018; Armentano, et al., Proc. NatI. Acad. Sci. USA (1990) 87:61416145; Huber, et al., Proc. NatI. Acad. Sci. USA (1991) 88:8039-8043; Ferry, et al., Proc. NatI. Acad. Sci. USA (1991) 88:8377-8381; Chowdhury, et al., Science (1991) 254:1802-1805; van Beusechem., et al., Proc. Nad. Acad. Sci. USA (1992) 89:7640-19 ; Kay, et al., Human Gene Therapy (1992) 3:641-647; Dai, et al., Proc. Natl.Acad. Sci. USA (1992) 89:10892-10895; Hwu, et al., J. Immunol. (1993) 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573). Recombinant retroviral vectors capable of transducing and expressing genes inserted into the genome of a cell can be produced by transfecting the recombinant retroviral genome into suitable packaging cell lines such as PA317 and Psi-CRIP (Comette et al., Human Gene Therapy (1991) 2:5-10; and Cone, et al., Proc. Natl. Acad. Sci. USA (1984) 81:6349). Recombinant adenoviral vectors can be used to infect a wide variety of cells and tissues in susceptible hosts (e.g., rat, hamster, dog, and chimpanzee) (Hsu et al., J. Infectious Disease, (1992) 166:769), and also have the advantage of not requiring mitotically active cells for infection.

The promoter driving dsRNA expression in either a DNA plasmid or viral vector of the invention may be a eukaryotic RNA polymerase I (e.g. ribosomal RNA promoter), RNA polymerase II (e.g. CMV early promoter or actin promoter or U1 snRNA promoter) or preferably RNA polymerase III promoter (e.g. U6 snRNA or 7SK RNA promoter) or a prokaryotic promoter, for example, the T7 promoter, provided the expression plasmid also encodes T7 RNA polymerase required for transcription from a T7 promoter. The promoter can also direct transgene expression to the pancreas (see, e.g., the insulin regulatory sequence for pancreas (Bucchini, et al., Proc. Natl. Acad. Sci. USA (1986) 83:2511-2515)).

In addition, expression of the transgene can be precisely regulated, for example, by using an inducible regulatory sequence and expression systems such as a regulatory sequence that is sensitive to certain physiological regulators, e.g., circulating glucose levels, or hormones (Docherty, et al., FASEB J. (1994) 8:20-24). Such inducible expression systems, suitable for the control of transgene expression in cells or in mammals include regulation by ecdysone, by estrogen, progesterone, tetracycline, chemical inducers of dimerization, and isopropyl-beta-D1-thiogalactopyranoside (EPTG). A person skilled in the art would be able to choose the appropriate regulatory/promoter sequence based on the intended use of the dsRNA transgene.

Preferably, recombinant vectors capable of expressing dsRNA molecules are delivered as described below, and persist in target cells. Alternatively, viral vectors can be used that provide for transient expression of dsRNA molecules. Such vectors can be repeatedly administered as necessary. Once expressed, the cFLIP dsRNAs bind to target cFLIP RNA and modulate its function or expression. Delivery of cFLIP dsRNA expressing vectors can be systemic, such as by intravenous or intramuscular administration, by administration to target cells ex-planted from the patient followed by reintroduction into the patient, or by any other means that allows for introduction into a desired target cell.

cFLIP dsRNA expression DNA plasmids are typically transfected into target cells as a complex with cationic lipid carriers (e.g. Oligofectamine™) or non-cationic lipid-based carriers (e.g. Transit-TKO™). Multiple lipid transfections for dsRNA-mediated knockdowns targeting different regions of a single target gene or multiple target genes over a period of a week or more are also contemplated by the present invention. Successful introduction of the vectors of the invention into host cells can be monitored using various known methods. For example, transient transfection can be signaled with a reporter, such as a fluorescent marker, such as Green Fluorescent Protein (GFP). Stable transfection of ex vivo cells can be ensured using markers that provide the transfected cell with resistance to specific environmental factors (e.g., antibiotics and drugs), such as hygromycin B resistance.

The cFLIP dsRNA molecules can also be inserted into vectors and used as gene therapy vectors for human pancreatic cancer patients. Gene therapy vectors can be delivered to a subject by, for example, intravenous injection, local administration (see U.S. Pat. No. 5,328,470) or by stereotactic injection (see e.g., Chen, et al., Proc. Natl. Acad. Sci. USA (1994) 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

### Example 4

### Method of Determining an Effective Dose of a cFLIP dsRNA

A therapeutically effective amount of a composition containing a sequence that encodes cFLIP specific dsRNA, (i.e., an effective dosage), is an amount that inhibits expression of the polypeptide encoded by the cFLIP target gene by at least 10 percent. Higher percentages of inhibition, e.g., 15, 20, 30, 40, 50, 75, 85, 90 percent or higher may be preferred in certain embodiments. Exemplary doses include milligram or microgram amounts of the molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). The compositions can be administered once per day, or in small subdoses throughout the day. The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of a composition can include a single treatment or a series of treatments. In some cases transient expression of the dsRNA may be desired. When an inducible promoter is included in the construct encoding an dsRNA, expression is assayed upon delivery to the subject of an appropriate dose of the substance used to induce expression.

Appropriate doses of a composition depend upon the potency of the molecule (the sequence encoding the dsRNA) with respect to the expression or activity to be modulated. One or more of these molecules can be administered to an animal (e.g., a human) to modulate expression or activity of one or more target polypeptides. A physician may, for example, prescribe a relatively low dose at first, subsequently increasing the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The efficacy of treatment can be monitored either by measuring the amount of the cFLIP target gene mRNA (e.g. using real time PCR) or the amount of polypeptide encoded by the target gene mRNA (Western blot analysis). In addition, the attending physician will monitor the symptoms associated with cancer afflicting the patient and compare with those symptoms recorded prior to the initiation of dsRNA treatment.

We claim:

1. A double-stranded ribonucleic acid (dsRNA) for inhibiting the expression of a cellular FLICE-like inhibitory protein (cFLIP) gene in a cell, wherein the dsRNA comprises a complementary RNA strand comprising a nucleotide sequence which is complementary to at least a part of the cFLIP gene.

2. The dsRNA of claim 1, further comprising a sense RNA strand, and wherein at least one end of said dsRNA comprises a nucleotide overhang of 1 to 4 nucleotides in length.

3. The dsRNA of claim 2, wherein the nucleotide overhang is 2 or 3 nucleotides in length.

4. The dsRNA of claim 2, wherein the nucleotide overhang is at the 3′-terminus of the complementary RNA strand.

5. The dsRNA of claim 4, wherein the dsRNA comprises a blunt end, wherein the blunt end is at the 5′-end of the complementary RNA strand.

6. The dsRNA of claim 1, wherein the nucleotide sequence is less than 25 nucleotides in length.

7. The dsRNA of claim 1, wherein the nucleotide sequence is 19 to 24 nucleotides in length.

8. The dsRNA of claim 1, wherein the nucleotide sequence is 20 to 24 nucleotides in length.

9. The dsRNA of claim 1, wherein the nucleotide sequence is 21 to 23 nucleotides in length.

10. The dsRNA of claim 1, wherein the nucleotide sequence is 22 or 23 nucleotides in length.

11. The dsRNA of claim 1, wherein the complementary RNA strand is less than 30 nucleotides in length.

12. The dsRNA of claim 1, wherein the complementary RNA strand is less than 25 nucleotides in length.

13. The dsRNA of claim 1, wherein the complementary RNA strand is 21 to 24 nucleotides in length.

14. The dsRNA of claim 1, wherein the complementary RNA strand is 23 nucleotides in length.

15. The dsRNA of claim 1, wherein the dsRNA further comprises a second (sense) RNA strand.

16. The dsRNA of claim 15, wherein the complementary RNA strand is 23 nucleotides in length and the second RNA strand is 21 nucleotides in length.

17. The dsRNA of claim 16, further comprising a blunt end and a nucleotide overhang of 2 nucleotides in length, wherein the nucleotide overhang is at the 3′-end of the complementary RNA strand and the blunt end is at the 5′-end of the complementary RNA strand.

18. The dsRNA of claim 1, wherein the nucleotide sequence of the complementary RNA strand is complementary to a primary or processed RNA transcript of the cFLIP gene.

19. The dsRNA of claim 15, wherein the complementary RNA strand comprises SEQ ID NO:2 and the second RNA strand comprises SEQ ID NO:1.

20. The dsRNA of claim 15, wherein the complementary RNA strand comprises SEQ ID NO:4 and the second RNA strand comprises SEQ ID NO:3.

21. The dsRNA of claim 15, wherein the complementary RNA strand comprises SEQ ID NO:7 and the second RNA strand comprises SEQ ID NO:1.

22. The dsRNA of claim 15, wherein the complementary RNA strand comprises SEQ ID NO:8 and the second RNA strand comprises SEQ ID NO:3.

23. A method for inhibiting the expression of a cellular FLICE-like inhibitory protein (cFLIP) gene in a cell, the method comprising:
(a) introducing into the cell a double-stranded ribonucleic acid (dsRNA), wherein the dsRNA comprises a complementary RNA strand comprising a nucleotide sequence which is complementary to at least a part of the cFLIP gene; and
(b) maintaining the cell produced in step (a) for a time sufficient to obtain degradation of a mRNA transcript of the cFLIP gene, thereby inhibiting expression of the cFLIP gene in the cell.

24. The method of claim 23, further comprising a second (sense) RNA strand.

25. The method of claim 24, wherein at least one end of the dsRNA comprises a nucleotide overhang of 1 to 4 nucleotides in length.

26. The method of claim 25, wherein the nucleotide overhang is 2 or 3 nucleotides in length.

27. The method of claim 25, wherein the nucleotide overhang is at the 3′-terminus of the complementary RNA strand.

28. The method of claim 27, wherein the dsRNA further comprises a blunt end, and wherein the blunt end is at the 5′-end of the complementary RNA strand.

29. The method of claim 23, wherein the nucleotide sequence is less than 25 nucleotides in length.

30. The method of claim 23, wherein the nucleotide sequence is 19 to 24 nucleotides in length.

31. The method of claim 23, wherein the nucleotide sequence is 20 to 24 nucleotides in length.

32. The method of claim 23, wherein the nucleotide sequence is 21 to 23 nucleotides in length.

33. The method of claim 23, wherein the nucleotide sequence is 22 or 23 nucleotides in length.

34. The method of claim 23, wherein the complementary RNA strand is less than 30 nucleotides in length.

35. The method of claim 23, wherein the complementary RNA strand is less than 25 nucleotides in length.

36. The method of claim 23, wherein the complementary RNA strand is 21 to 24 nucleotides in length.

37. The method of claim 23, wherein the complementary RNA strand is 23 nucleotides in length.

38. The method of claim 23, wherein the dsRNA further comprises a second (sense) RNA strand.

39. The method of claim 38, wherein the complementary RNA strand is 23 nucleotides in length and the second RNA strand is 21 nucleotides in length.

40. The method of claim 39, wherein the dsRNA comprises a blunt end and a nucleotide overhang of 2 nucleotides in length, wherein the complementary RNA strand further comprises a 3′-end and a 5′-end, and wherein the nucleotide overhang is at the 3′-end of the complementary RNA strand and the blunt end is at the 5′-end of the complementary RNA strand.

41. The method of claim 23, wherein the nucleotide sequence of the complementary RNA strand is complementary to a primary or processed RNA transcript of the cFLIP gene.

42. The method of claim 24, wherein the complementary RNA strand comprises SEQ ID NO:2 and the second RNA strand comprises SEQ ID NO:1.

43. The method of claim 24, wherein the complementary RNA strand comprises SEQ ID NO:4 and the second RNA strand comprises SEQ ID NO:3.

44. The method of claim 24, wherein the complementary RNA strand comprises SEQ ID NO:7 and the second RNA strand comprises SEQ ID NO:1.

45. The method of claim 24, wherein the complementary RNA strand comprises SEQ ID NO:8 and the second RNA strand comprises SEQ ID NO:3.

46. The method of claim 23, wherein the cell is a tumor cell.

47. The method of claim 46, wherein the tumor cell is resistant to treatment with an apoptosis-inducing drug.

48. The method of claim 47, wherein the apoptosis-inducing drug is TRAIL.

49. A pharmaceutical composition for improving the effectiveness of an apoptosis-inducing drug in a mammal, comprising a dsRNA and a pharmaceutically acceptable carrier, wherein the dsRNA comprises a complementary RNA strand comprising a complementary nucleotide sequence which is complementary to at least a part of a cellular FLICE-like inhibitory protein (cFLIP) gene.

50. The pharmaceutical composition of claim 49, wherein the apoptosis-inducing drug is a tumor necrosis factor (TNF) or a TNF-related ligand.

51. The pharmaceutical composition of claim 50, wherein the TNF-related ligand is selected from the group of ligands consisting of a TRAMP ligand, a CD95 ligand, a TNFR-1 ligand, and a TNF-related apoptosis-inducing ligand (TRAIL).

52. The pharmaceutical composition of claim 50, wherein the TFN-related ligand is TRAIL.

53. The pharmaceutical composition of claim 49, wherein the dsRNA further comprises a nucleotide overhang of 1 to 4 nucleotides in length.

54. The pharmaceutical composition of claim 53, wherein the nucleotide overhang is at the 3′-terminus of the complementary RNA strand.

55. The pharmaceutical composition of claim 49, wherein the nucleotide sequence is less than 25 nucleotides in length.

56. The pharmaceutical composition of claim 49, wherein the nucleotide sequence is 19 to 24 nucleotides in length.

57. The pharmaceutical composition of claim 49, wherein the nucleotide sequence is 20 to 24 nucleotides in length.

58. The pharmaceutical composition of claim 49, wherein the nucleotide sequence is 21 to 23 nucleotides in length.

59. The pharmaceutical composition of claim 49, wherein the nucleotide sequence is 22 or 23 nucleotides in length.

60. The pharmaceutical composition of claim 49, wherein the complementary RNA strand is less than 30 nucleotides in length.

61. The pharmaceutical composition of claim 49, wherein the complementary RNA strand is less than 25 nucleotides in length.

62. The pharmaceutical composition of claim 49, wherein the complementary RNA strand is 21 to 24 nucleotides in length.

63. The pharmaceutical composition of claim 49, wherein the complementary RNA strand is 23 nucleotides in length.

64. The pharmaceutical composition of claim 49, wherein the dsRNA further comprises a second (sense) RNA strand.

65. The pharmaceutical composition of claim 64, wherein the complementary RNA strand is 23 nucleotides in length and the second RNA strand is 21 nucleotides in length.

66. The pharmaceutical composition of claim 65, wherein the dsRNA comprises a blunt end and a nucleotide overhang of 2 nucleotides in length, wherein the complementary RNA strand comprises a 3′-end and a 5′-end, and wherein the nucleotide overhang is at the 5′-end of the complementary RNA strand and the blunt end is at the 3′-end of the complementary RNA strand.

67. The pharmaceutical composition of claim 49, wherein the nucleotide sequence of the complementary RNA strand is complementary to a primary or processed RNA transcript of the cFLIP gene.

68. The pharmaceutical composition of claim 64, wherein the complementary RNA strand comprises SEQ ID NO:2 and the second RNA strand comprises SEQ ID NO:1.

69. The pharmaceutical composition of claim 64, wherein the complementary RNA strand comprises SEQ ID NO:4 and the second RNA strand comprises SEQ ID NO:3.

70. The pharmaceutical composition of claim 64, wherein the complementary RNA strand comprises SEQ ID NO:7 and the second RNA strand comprises SEQ ID NO:1.

71. The pharmaceutical composition of claim 64, wherein the complementary RNA strand comprises SEQ ID NO:8 and the second RNA strand comprises SEQ ID NO:3.

72. The pharmaceutical composition of claim 49, wherein the mammal is a human.

73. The pharmaceutical composition of claim 49, wherein the dosage unit of dsRNA is less than 5 milligram of dsRNA per kilogram body weight of the mammal.

74. The pharmaceutical composition of claim 49, wherein the dosage unit of dsRNA is in a range of 0.01 to 2.5 milligrams, 0.1 to 200 micrograms, or 0.1 to 100 micrograms per kilogram body weight of the mammal.

75. The pharmaceutical composition of claim 49, wherein the dosage unit of dsRNA is less than 50 micrograms of dsRNA per kilogram body weight of the mammal.

76. The pharmaceutical composition of claim 49, wherein the dosage unit of dsRNA is less than 25 micrograms per kilogram body weight of the mammal.

77. The pharmaceutical composition of claim 49, wherein the pharmaceutically acceptable carrier is an aqueous solution.

78. The pharmaceutical composition of claim 77, wherein the aqueous solution is phosphate buffered saline.

79. The pharmaceutical composition of claim 49, wherein the pharmaceutically acceptable carrier comprises a micellar structure selected from the group consisting of a liposome, capsid, capsoid, polymeric nanocapsule, and polymeric microcapsule.

80. The pharmaceutical composition of claim 79, wherein the micellar structure is a liposome.

81. The pharmaceutical composition of claim 49, wherein the pharmaceutical composition is formulated for administration by inhalation, oral ingestion, infusion or injection.

82. The pharmaceutical composition of claim 49, wherein the composition is formulated for administration by intravenous, intraparenteral, or intratumoral infusion or injection.

83. A method for improving the effectiveness of a bioactive substance that induces receptor-mediated apoptosis in a tumor cell in a mammal, which comprises administering to said mammal a pharmaceutical composition comprising a double-stranded ribonucleic acid (dsRNA) and a pharmaceutically acceptable carrier, wherein the dsRNA comprises a complementary RNA strand comprising a complementary nucleotide sequence which is complementary to at least a part of a cellular FLICE-like inhibitory protein (cFLIP) gene.

84. The method of claim 83, wherein the bioactive substance is a tumor necrosis factor (TNF) or a TNF-related ligand.

85. The method of claim 84, wherein the TNF-related ligand is selected from the group of ligands consisting of a TRAMP ligand, a CD95 ligand, a TNFR-1 ligand, and a TNF-related apoptosis-inducing ligand (TRAIL).

86. The method of claim 84, wherein the TNF-related ligand is TRAIL.

87. A method for treating cancer in a mammal, the method comprising:
a) administering to the mammal a pharmaceutical composition comprising a double-stranded ribonucleic acid (dsRNA), wherein the dsRNA comprises a complementary RNA strand comprising a nucleotide sequence which is complementary to at least a part of a cellular FLICE-like inhibitory protein (cFLIP) gene; and
(b) administering to the mammal a pharmaceutical composition comprising a bioactive substance that induces receptor-mediated apoptosis in a tumor cell.

88. The method of claim 87, wherein the bioactive substance is a tumor necrosis factor (TNF) or a TNF-related ligand.

89. The method of claim 88, wherein the TNF-related ligand is selected from the group of ligands consisting of a TRAMP ligand, a CD95 ligand, a TNFR-1 ligand, and a TNF-related apoptosis-inducing ligand (TRAIL).

90. The method of claim 88, wherein the TNF-related ligand is TRAIL.

91. The method of claim 87, wherein the dsRNA and the bioactive substance are administered together in one pharmaceutical composition.

92. A pharmaceutical composition for inhibiting the expression of a cellular FLICE-like inhibitory protein (cFLIP) in a mammal, comprising a dsRNA and a pharmaceutically acceptable carrier, wherein the dsRNA comprises a complementary RNA strand comprising a complementary nucleotide sequence which is complementary to at least a part of the cFLIP gene.

93. The pharmaceutical composition of claim 92, further comprising an apoptosis-inducing drug.

94. The pharmaceutical composition of claim 93, wherein the apoptosis-inducing drug is a tumor necrosis factor (TNF) or a TNF-related ligand.

95. The pharmaceutical composition of claim 94, wherein the TNF-related ligand is selected from the group of ligands consisting of a TRAMP ligand, a CD95 ligand, a TNFR-1 ligand, and a TNF-related apoptosis-inducing ligand (TRAIL).

96. The pharmaceutical composition of claim 94, wherein the TNF-related ligand is TRAIL.
